(19) United States
(12) Patent Application Publication
(10) Pub. No.: US 2006/0058583 A1
(43) Pub. Date: Mar. 16, 2006

(54) Lubricant for endoscope and endoscope

(76) Inventors: Jun Matsumoto, Hino-shi (JP); Hideyuki Ito, Tama-shi (JP)

Correspondence Address: OSTROLENK FABER GERB & SOFFEN, 1180 AVENUE OF THE AMERICAS, NEW YORK, NY, 100368403, US

(21) Appl. No.: 11/227,818

(22) Filed: Sep. 15, 2005

(30) Foreign Application Priority Data
Sep. 16, 2004 (JP) 2004-269970

(51) Int. Cl. A61B 1/00

(52) U.S. Cl. 600/153; 600/101; 600/133

(57) ABSTRACT

A lubricant for an endoscope, which is designed to be applied to surfaces of built-in members for enhancing lubricity among a plurality of built-in members accommodated in a flexible insertion tube constituting an insertion portion of endoscope, the lubricant including a porous carbonaceous material.

### CROSS-REFERENCE TO RELATED APPLICATIONS

This application is based upon and claims the benefit of priority from prior Japanese Patent Application No. 2004-269970, filed Sep. 16, 2004, the entire contents of which are incorporated herein by reference.

### BACKGROUND OF THE INVENTION

1. Field of the Invention

The present invention relates to a lubricant for an endoscope and to an endoscope where a lubricant is employed, and in particular, to a lubricant for an endoscope which is excellent in resistance to autoclave sterilization.

2. Description of the Related Art

The insertion portion of an endoscope which is adapted to be introduced into a body cavity is designed to accommodate therein various kinds of members such as a fiber bundle for transmitting an image, a fiber bundle for transmitting light, a wire for angulation, air-supply/water-supply tubes, etc. When this insertion portion is caused to bend with these built-in members being accommodated therein, the built-in members are caused to contact each other, giving rise to problems such as breaking of the fiber for instance.

In view of solving such problems, there has been proposed to interpose molybdenum disulfide as a lubricant between the built-in members.

Meanwhile, since the endoscope is designed to be repeatedly used, the endoscope is required to be washed and sterilized every time after use. In recent years, in order to prevent patient-to-patient infection through an endoscope, it is now made it a rule to perform the sterilization of the insertion portion of an endoscope by making use of a peroxide-based disinfectant having a very strong bactericidal action.

However, when the insertion portion of an endoscope is subjected to sterilization by making use of this peroxide, sulfide is caused to generate through the reaction between the peroxide and molybdenum disulfide employed as a lubricant, thereby degrading, due to this sulfide, the aforementioned built-in members as well as the resin constituting the flexible tube of endoscope.

In order to solve such a problem, there has been proposed to employ, in place of molybdenum disulfide, other kinds of lubricants such as carbon graphite, boron nitride (BN), polytetrafluoroethylene (PTFE), fluorinated oil, fluorinated grease, etc. (see for example, JP Laid-open Patent Publication (Kokai) No. 11-28184 (1999)).

However, when these lubricants are employed in an endoscope for digestive organ examination, the lubricating performance thereof is caused to deteriorate in a long term employment thereof, resulting in an increase in quantity of angling force and hence in the infliction of damage to bending members such as an angle wire.

Further, in recent years, autoclave sterilization in a high-pressure steam atmosphere, which is excellent in sterilization action, has been widely employed. In order to make an endoscope for digestive organ examination applicable to such autoclave sterilization, it is required to develop a lubricant which is excellent in lubricity and hardly oxidizable by high-temperature/high-pressure steam.

### BRIEF SUMMARY OF THE INVENTION

An object of the present invention is to provide a lubricant for an endoscope, which is capable of keeping lubricity among built-in members accommodated in a bending portion of the flexible tube of an endoscope even if the endoscope is subjected to autoclave sterilization.

Another object of the present invention is to provide an endoscope where such a lubricant is employed.

According to one aspect of the present invention, there is provided a lubricant for an endoscope, which is designed to be applied to surfaces of built-in members for enhancing lubricity among a plurality of built-in members accommodated in a flexible insertion tube constituting an insertion portion of endoscope, the lubricant being formed of a porous carbonaceous material.

According to another aspect of the present invention, there is provided an endoscope comprising a flexible insertion tube constituting an insertion portion of endoscope, and a plurality of built-in members accommodated in the flexible insertion tube and coated, on the surfaces thereof, with a layer of lubricant formed of a porous carbonaceous material for enhancing lubricity among the built-in members.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a cross-sectional view showing a flexible insertion tube constituting an insertion portion of an endoscope where a lubricant according to one embodiment of the present invention is employed; and

FIG. 2 is a cross-sectional view showing a glass fiber to be built into the flexible insertion tube shown in FIG. 1.

### DETAILED DESCRIPTION OF THE INVENTION

The lubricant for endoscope according to one aspect of the present invention is designed to be employed for enhancing the lubricity among a plurality of built-in members accommodated in a flexible insertion tube constituting the insertion portion of an endoscope and is featured in that it comprises a porous carbonaceous material.

This porous carbonaceous material is excellent, as a lubricant, in many respects such as adhesion to metal, adhesion to fiber, lubricity, corrosion resistance, and heat resistance, and is especially excellent in resistance to autoclave sterilization in a high-pressure steam atmosphere. Therefore, this porous carbonaceous material is very suited for use as a lubricant to be employed in a bending portion or insertion portion of an endoscope.

As for specific examples of this porous carbonaceous material, it is possible to employ those which can be obtained through carbonization of bran, examples of which include rice bran and wheat bran. Other than these brans, it is also possible to employ rice hull, buckwheat husks, soy bean husk, gluten feed, and various kinds of fine powder which can be created in the course of processing grains.

The carbonization of bran can be performed by baking it at a predetermined temperature in an inert or non-oxidizing atmosphere such as nitrogen or argon. Although there is not any particular limitation with regard to the baking temperature, it should preferably be 500° C. or more, more preferably 700° C. or more.

The porous carbonaceous material prepared through the carbonization of these raw materials is especially excellent in adhesion to metal or to a fiber and also excellent in resistance such that it can be hardly oxidized even if it is subjected to autoclave sterilization in a high-pressure steam atmosphere.

As one example of the porous carbonaceous material, it is possible to employ those which can be prepared in such a way that bran is mixed at first with a thermosetting resin such as phenol resin to obtain a mixture which is then molded, carbonized and pulverized to form the porous carbonaceous material. Since the thermosetting resin in these porous carbonaceous materials is capable of functioning as a binder, these porous carbonaceous materials to be obtained are excellent in homogeneity and formed of desired grain size, thus exhibiting excellent lubricity.

As for the particle diameter of porous carbonaceous material powder, it should preferably be confined within the range of 0.5 to 150 μm. The porous carbonaceous material powder having such a limited particle diameter is advantageous in that it is possible to enhance the lubricity of the built-in members inserted in the flexible insertion tube without inflicting any damage to them.

The lubricant for endoscope according to one aspect of the present invention, as explained above, is excellent in many respects such as adhesion to metal, adhesion to fiber, lubricity, corrosion resistance, and heat resistance, and is especially excellent in that even if it is subjected to autoclave sterilization, it is possible to retain the lubricity inside the bending portion of an endoscope.

The endoscope according to another aspect of the present invention is featured in that it comprises a flexible insertion tube constituting an insertion portion of an endoscope, and a plurality of built-in members accommodated in the flexible insertion tube and coated, on the surfaces thereof, with a layer of lubricant formed of a porous carbonaceous material for enhancing lubricity among the built-in members.

In the case of this endoscope, it is possible to prevent any increase in quantity of angling force even if the flexible insertion tube of the insertion portion of an endoscope is repeatedly bent and hence to prevent the infliction of damage to bending members such as an angle wire. Further, even if this endoscope is subjected to autoclave sterilization in a high-temperature/high pressure steam atmosphere, the lubricity of the lubricant would not be deteriorated.

In this case, the lubricant layer can be formed also on the inner surface of the flexible insertion tube, thereby making it possible to further enhance the function of lubricant.

Next, the best mode for carrying out the present invention will be explained.

The lubricant for endoscope according to one aspect of the present invention is a lubricant which is designed to be applied for enhancing lubricity among a plurality of built-in members accommodated in a flexible insertion tube constituting an insertion portion of endoscope, the lubricant comprising a porous carbonaceous material.

This porous carbonaceous material is excellent in many respects such as adhesion to metal, adhesion to fiber, lubricity, corrosion resistance, and heat resistance, and is especially excellent in that even if it is subjected to autoclave sterilization, it cannot be oxidized and hence is capable of retaining the lubricity thereof. Therefore, this porous carbonaceous material is very suited for use as a lubricant for endoscope.

As for specific examples of the lubricant which are very suited for use in the first aspect of the present invention, it is possible to employ porous carbonaceous materials which are set forth in JP Laid-open Patent Publication (Kokai) No. 10-101453 (1998). These porous carbonaceous materials can be manufactured by a method which comprises the steps of: kneading bran such as rice bran or wheat bran together with a thermosetting resin and a suitable quantity of a water or an aqueous solution containing binder to form a kneaded mixture comprising the bran and thermosetting resin, etc.; granulating the mixture to form a granular material having a predetermined range of particle size; introducing the granular material into a desired mold; molding the granular material while compressing and degassing the granular material to form a molded product; releasing the molded product from the mold and baking the molded product in an inert gas atmosphere or in vacuum by heating it up to a desired ultimate baking temperature according to a predetermined heating rate to thereby carbonize the molded product; and cooling the molded product down to normal temperature from the ultimate baking temperature according to a predetermined cooling rate.

This porous carbonaceous material is formed of a stereostructure including a large number of cells dispersed therein, each cell being relatively flat and clear-cut in configuration, some of the cells being formed into a sponge-like structure and many of the cells being respectively surrounded by a dense carbon material which is irregularly curved, thus forming a continuous or laminar structure as a whole.

The porous carbonaceous material which has been baked at a temperature of 500° C. or more is featured in that it is very low in friction coefficient and very excellent as a lubricant. Further, this porous carbonaceous material is excellent in heat resistance, can be hardly oxidized even in a high-temperature/high-pressure steam atmosphere, and is also excellent in corrosion resistance. Moreover, this porous carbonaceous material is very excellent in adhesion to metal and glass.

Because of these reasons, when the porous carbonaceous materials set forth in JP Laid-open Patent Publication (Kokai) No. 10-101453 are applied to the surfaces of built-in members accommodated in a flexible insertion tube constituting an insertion portion of endoscope, they will exhibit very excellent performance as a lubricant for enhancing the lubrication among the built-in members.

One of preferable examples of the porous carbonaceous material would be RB ceramics (trade name: Sanwa Yushi Co., Ltd.).

Next, a specific embodiment of the present invention will be explained.

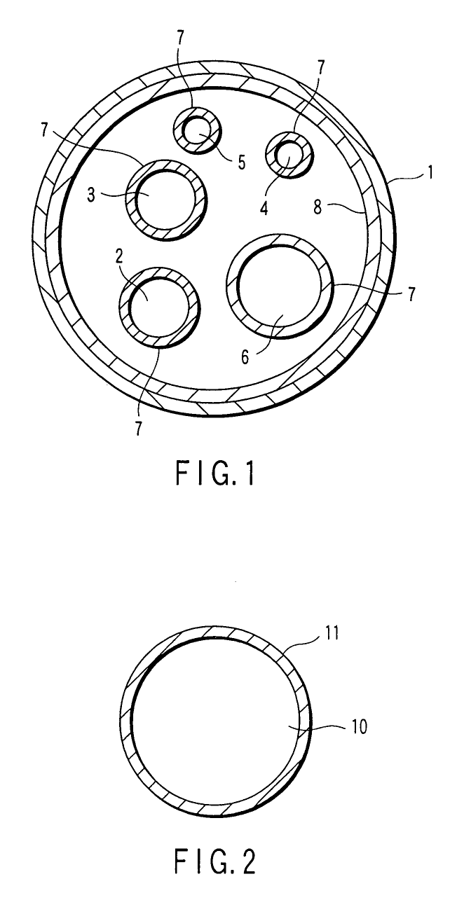

FIG. 1 is a cross-sectional view showing a flexible insertion tube constituting an insertion portion of an endoscope according to one aspect of the present invention. Referring to FIG. 1, various kinds of members such as a light guide bundle 2, an image guide bundle 3, an air-supply tube 4, a water-supply tube 5, and a channel tube 6 are introduced into a flexible insertion tube 1, thereby assembling an endoscope. Incidentally, the light guide bundle 2 and the image guide bundle 3 are respectively formed of a bundle of glass fibers which are covered by a sheath tube made of silicone rubber.

The outer surfaces of these built-in members are respectively covered by a lubricating material layer 7 formed of a porous carbonaceous material. Incidentally, in the embodiment shown in FIG. 1, the inner surface of the flexible insertion tube 1 is also covered by a lubricating material layer 8.

FIG. 2 is a cross-sectional view showing a piece of glass fiber constituting the light guide bundle 2 and the image guide bundle 3 shown in FIG. 1. Not only the outer surfaces of the light guide bundle 2 and the image guide bundle 3, but also the outer surface of the glass fiber 10 constituting these bundles is covered by a lubricating material layer 11. These lubricating material layers 7, 8 and 11 can be applied to these outer surfaces by various methods.

For example, the powder of porous carbonaceous material may be directly applied to the outer surfaces of these built-in members or to the inner surface of the flexible insertion tube 1, thereby covering these surfaces with a porous carbonaceous material. Alternatively, the powder of a porous carbonaceous material may be dissolved in a suitable kind of dispersing medium (for example, disperse it in a fluorinated solvent) to obtain a dispersion, which is then sprayed to the outer surfaces of these built-in members or to the inner surface of the flexible insertion tube 1. Alternatively, the powder of a porous carbonaceous material may be applied to the outer surfaces of these built-in members or to the inner surface of the flexible insertion tube 1 by way of baking using a suitable kind of binder such as epoxy resin or phenolic resin.

In this manner, these lubricating material layers 7, 8 and 11, each having a thickness of 1 to 150 μm, can be formed on the outer surfaces of these built-in members and on the inner surface of the flexible insertion tube 1.

Next, specific examples of the present invention will be explained.

### EXAMPLE 1

As a powder lubricant, a porous carbonaceous material powder (RB ceramics (trade name): Sanwa Yushi Co., Ltd.) was coated on the outer surfaces of built-in members of flexible insertion tube, i.e. the outer surfaces of a light guide bundle, an image guide bundle, an air-supply tube, a water-supply tube and a channel tube by means of dry process. The powder lubricant thus coated was then rubbed on the outer surfaces of these built-in members and the excess amount of the powder lubricant was removed from the outer surfaces of these built-in members. Likewise, the powder lubricant was also coated on the inner surface of the flexible insertion tube and then these built-in members were inserted into the flexible insertion tube, thereby assembling an endoscope.

### COMPARATIVE EXAMPLES 1-3

The procedures of Example 1 were repeated in the same manner to thereby coat the powder lubricant on the outer surfaces of these built-in members as well as on the inner surface of the flexible insertion tube except that molybdenum disulfide, boron nitride and carbon graphite were respectively substituted, as a powder lubricant, for RB ceramics employed in Example 1. Then, these built-in members were inserted into the flexible insertion tube, thereby assembling the endoscope.

RB ceramics, molybdenum disulfide, boron nitride and carbon graphite employed in Example 1 and Comparative Examples 1-3 were evaluated with respect to the adhesion to metal, adhesion to fiber, lubricity, corrosion resistance and heat resistance thereof.

The methods of tests on these characteristics were as follows.

Adhesion to Metal:

Each of aforementioned lubricants was dry-coated on a metal plate and then, by making use of Try Gear TYPE: HEIDON-22H as a surface tester, a test was performed to find out if the lubricant could be peeled away from the metal plate and friction coefficient would be caused to increase in an oscillating sliding test.

Adhesion to Fiber:

By making use of each of the aforementioned lubricants, a light guide bundle was prepared and then a test was performed to find out if the quantity of light would be caused to decrease due to the break of fiber as a result of repeated bending of the light guide bundle to cause rubbing among the fibers.

Lubricity:

By making use of each of the aforementioned lubricants, angling force was measured.

Corrosion Resistance:

Each of aforementioned lubricants was introduced into a hydrogen peroxide gas atmosphere and then investigation was performed, by means of FT-IR, to see if the components of the lubricant were denatured.

Heat Resistance:

Each of the aforementioned lubricants was introduced into an atmosphere heated to a temperature ranging from 300 to 800° C. and then investigation was performed to see how much the initial lubricity of lubricants could be sustained.

The results are shown in the following Table.

TABLE

| | Molybdenum | Boron | | RB |
|---|---|---|---|---|
| | disulfide | nitride | Graphite | ceramics |
| Adhesion to metal | ⊚ | X | Δ | ⊚ |
| Adhesion to fiber | ⊚ | X | X | ⊚ |
| Lubricity | Friction | Friction | Friction | Friction |
| | coefficient | coefficient | coefficient | coefficient |
| | (0.1) | (0.2) | (0.1) | (0.15) |
| | ⊚ | Δ | ⊚ | ◯ |
| Corrosion | Generation of | ◯ | ◯ | ◯ |
| resistance | sulfur oxide | | | |
| | X | | | |
| Heat resistance | 350° C. | 600° C. | 800° C. | 800° C. |

Incidentally, in this Table, the double circle symbol means “very excellent”, single circle means “excellent”, delta means “ordinary”, and x means poor”.

It will be seen from Table 1 that RB ceramics indicated excellent results in all characteristics. Whereas, in the case of molybdenum disulfide, although excellent results were obtained with respect to adhesion to metal, adhesion to fiber, and lubricity, the corrosion resistance test thereof indicated a poor result due to the generation of sulfur oxide and the heat resistance thereof was also very low. Further, the cases of boron nitride and carbon graphite were all inferior with respect to the adhesion to metal and the adhesion to fiber.

### EXAMPLE 2

A porous carbonaceous material powder was dispersed in a fluorinated solvent employed as a dispersing medium to obtain 1-10% dispersion, which was sprayed to the surfaces of built-in members of flexible insertion tube, i.e. the outer surfaces of a light guide bundle, an image guide bundle, an air-supply tube, a water-supply tube and a channel tube. An excess amount of the dispersion was removed from the outer surfaces of these built-in members. The dispersion was introduced into and passed through the flexible insertion tube to coat a thin layer of porous carbonaceous material on the inner surface of the flexible insertion tube and then these built-in members were inserted into the flexible insertion tube, thereby assembling an endoscope.

When the flexible insertion tube of insertion portion of endoscope thus manufactured was repeatedly bent, it was possible to prevent any increase in angling force and the generation of damage could not be found in the angle wire as well as in other bending members.

Further, when the endoscope provided with these flexible tubes was subjected to autoclave sterilization for 5 minutes at a temperature of 135° C. in a steam atmosphere of 2 atm, the oxidation of the lubricant could not be recognized.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A lubricant for an endoscope, which is designed to be applied to surfaces of built-in members for enhancing lubricity among a plurality of built-in members accommodated in a flexible insertion tube constituting an insertion portion of the endoscope, the lubricant comprising a porous carbonaceous material.

2. The lubricant according to claim 1, wherein the porous carbonaceous material is one which is formed through carbonization of bran.

3. The lubricant according to claim 2, wherein the bran is at least one material selected from the group consisting of rice bran, wheat bran, rice hull, buckwheat husks, soy bean husk and gluten feed.

4. The lubricant according to claim 1, wherein the porous carbonaceous material is one which is formed through carbonization of a mixture comprising bran and a thermosetting resin.

5. The lubricant according to claim 1, wherein the porous carbonaceous material is formed of particles having a particle diameter ranging from 0.5 μm to 150 μm.

6. An endoscope which comprises:
a flexible insertion tube constituting an insertion portion of endoscope; and
a plurality of built-in members accommodated in the flexible insertion tube and coated, on the surfaces thereof, with a layer of lubricant formed of a porous carbonaceous material for enhancing lubricity among the built-in members.

7. The endoscope according to claim 6, wherein the layer of lubricant is also formed on an inner surface of the flexible insertion tube.

8. The endoscope according to claim 6, wherein said plurality of built-in members include at least one member selected from the group consisting of a light guide bundle, an image guide bundle, an air-supply tube, a water-supply tube and a channel tube.

9. The endoscope according to claim 6, wherein the layer of lubricant is formed by a method wherein a porous carbonaceous material powder is directly applied to a surface of the member, by a method wherein a porous carbonaceous material is dissolved in a dispersing medium to obtain a dispersion, which is then sprayed to a surface of the member, or by a method wherein a porous carbonaceous material powder is applied to a surface of the member by way of baking together with a binder.

10. The endoscope according to claim 6, wherein the porous carbonaceous material is one which is formed through carbonization of bran.

11. The endoscope according to claim 10, wherein the bran is at least one material selected from the group consisting of rice bran, wheat bran, rice hull, buckwheat husks, soy bean husk and gluten feed.

12. The endoscope according to claim 6, wherein the porous carbonaceous material is one which is formed through carbonization of a mixture comprising bran and a thermosetting resin.

13. The endoscope according to claim 6, wherein the porous carbonaceous material is formed of particles having a particle diameter ranging from 0.5 μm to 150 μm.
